(19) United States
(12) Patent Application Publication
(10) Pub. No.: US 2009/0181073 A1
(43) Pub. Date: Jul. 16, 2009

(54) Method for treating irritation caused by hair removal

(76) Inventors: Stephen M. Renzin, Larchmont, NY (US); William Schmitt, Brandford, CT (US)

Correspondence Address: William W. Jones, 6 Juniper Lane, Madison, CT, 06443, US

(21) Appl. No.: 12/008,875

(22) Filed: Jan. 15, 2008

(51) Int. Cl. A61K 9/70; A61K 35/20; A61P 17/00

(52) U.S. Cl. 424/447; 424/535

(57) ABSTRACT

A method for relieving skin irritation caused by hair removal involves applying a chilled skim milk additive which to the irritated skin. The additive is applied to the skin on pads or similar carriers which are provided with a self-contained component that can be frozen or, when compressed and ruptured, will lower the temperature of the pads or carriers to provide the soothing cold. The skim milk can be incorporated into the pads or carriers in several different ways. One preferred way is to form a slurry of the dry skim milk in glycerin, which slurry is applied to the pads or carriers.

### TECHNICAL FIELD

This invention relates to a method for treating skin irritations which result from body hair removal procedures. More particularly, this invention relates to such a method which involves the application of cold combined with a dry milk product, such as dry skim milk, to the irritated skin after the hair removal procedure is completed. The milk and cold combination can be applied to the irritated skin by means of a pad which can be worn by the user during normal daily activity. The pads used can be similar to those shown and described in our co-pending patent application PCT/US2005/02572, the disclosure of which is encorporated herein in its entirety.

### BACKGROUND ART

Hair growth is a well understood biological process that has resulted in a wide variety of treatments of and removal of hair for either a cosmetic benefit to existing hair or removal of hair by a variety of treatments. The mature epithelial hair follicle has a hair bulb where the hair is created. There are two types of hairs, terminal hairs which are thick, pigmented and generally long and vellus hairs which are small in diameter, short and non-pigmented. Hair of both types grow over the entire skin surface except for the soles of the feet and palms of the hands.

Actively growing terminal follicles also transfer pigment to the matrix of the hair. The hair follicle is remarkably dynamic. The follicle cycles through periods of active growth (anagen phase), regression (catagen phase), and quiescence (telogen) phase. These cycles vary in length depending on where the hair is located. There is also significant gender-determined differences in where terminal hair grows with males having more facial and body hairs then females.

Humans and cultures have adopted various means of controlling their hair and in many cases removing hair by various means, most of which are temporary.

The problem with many forms of hair removal is that it can cause irritation of skin at the follicle and underlying hair bulb if removed by force. Various methods of hair removal can create considerable skin irritation in a very significant number of individuals. Examples of this irritation are painfully swollen and inflamed skin following hair removal on various areas of the body, which hair removal is accomplished by shaving, electrolysis, laser removal, depilatory usage, waxing, and other hair removal methods. The inflammation is sometimes apparent as a significant and persistent redness, small blisters as in burns, and sometimes an acne like reaction. Many persons complain that the post treatment remedies given by the individuals performing the hair removal procedures are not effective and can be quite expensive in that they are usually fancy creams of some sort.

All hair removal techniques can cause irritation to the areas where hair is removed. Shaving, waxing, tweezing, depilatories, electrolysis, laser and other hair removal methods often irritate the hair follicle to the point of causing pain, heat, redness, swelling and eruption like skin reactions. These reactions appear as irritating red bumps which at the very least are an embarrassing sight if not a stinging reminder lasting many hours or days. Left untreated, these can also be a source for infection. These symptoms occur because blood vessels in the area dialate to bring in extra white blood cells at the site of injury, which is a classical irritation reaction. As a result, dead cells begin to accumulate the affected area. Various methods for the removal of body hair are as follows.

Wet shaving is more popular and common than other methods of hair removal because it is easy, can be painless, and cheap. Another advantage is that the razor blade cuts hairs doser to the surface and leaves stubble of a uniform length, so blade shaving can produce a smooth, dose shave. A shaving angle of 28 to 32 degrees between the blade and the skin will give the closest shave with the least amount of irritation. It is a good option in extensive and well-keratinized corporal surface, like a woman's leg and a man's face. The razor has to be dean, and the blade sharp. Dull blades cause pain and can cause irritation.

The rate of growth varies with the area of the body and the age of the person. The beard, for example, grows more during the day than at night. Wet shaving may promote some problems, namely: skin irritations, small cuts, and the potential for local infections, like warts or impetigo. Excessive local pigmentation can occur when the beard is dark and hairs are arranged closely together and also for other reasons, as, for example, too close shaving or constant local irritation by frequently used photosensitizing pre-shave and after shave products. Ingrown hair is also a common complaint from wet shaving. Many factors can play a role. Shaving too dose or against the grain, especially in the neck area, can result in clipping off whiskers beneath the skin surface. This becomes worse when the hair is coarse and curly since they may have a tendency to curl back and reenter in the skin, causing an inflammation around the ingrown hair tip and result in scarring. Another factor is the roughness of the hair's surface after it has been cut. The rougher it is, the greater its chances of being caught, either along the side of the follicle or by the skin next to the follicle, becoming ingrown.

A waxing or sugaring treatment removes the hair from the area treated by pulling it out. In a professional treatment, first the hair is trimmed with a scissors so that the wax or sugar solution can reach the follicles. Then, using a wooden stick or appliance, a technician places warm wax or concentrated sugar solution on the area a little bit at a time. Cloth strips are placed over the hot wax or sugar and, after it hardens, it is pulled away from the skin. In the case of a Bikini wax, areas that show beyond a bathing suit are treated, in the case of a “Brazilian” the entire pubic region, except for a small patch just above the vaginal area is treated. Many women request a Brazilian because it gives a cleaner, closer hair removal and the freedom to wear even the most revealing swim wear and lingerie. Expect to be waxed to the max if you visit a salon for a Brazilian treatment. A traditional Brazilian includes the labia and the area that reaches into the buttocks. If there are stray hairs after waxing, the technician may also tweeze the area. The key to allowing wax to penetrate into the follicles (and provide the closest possible wax) is to use an experienced professional. Many men are getting waxing of the chest or back by the same treatment. It is normal to have very significant pain during the procedure and significant irritation during the first few days after a bikini or Brazilian wax. This is due to the clogging of pores and ripping out the hair bulb and follicles.

The main advantage of chemical depilatories is the fact they are painless and have a slower regrowth than shaving. They can give results that last up to two weeks, and the hair that regrows does not feel as bristly as after shaving. The depilatory must be left on the skin for about three to fifteen minutes, depending on the depilatory agent and the coarseness of the hair. All formulations act by disrupting disulfide bonds, especially those involving cystine, breaking the hair's protein structure. The hairs break-off at or slightly below the surface of the skin and can be wiped off. Depilatories are composed of detergents, hair-shaft-swelling agents, adhesives, pH adjusters, and bond-breaking agents. Detergents such as sodium lauryl sulfate, laureth-23, or laureth-4 remove the protective hair sebum and allow penetration of the bond-breaking agent. Further penetration is accomplished with swelling agents, such as urea or thiourea. Adhesives, such as paraffin allow the mixture to adhere to hairs, and adjustment of pH is important to minimize cutaneous irritation. Several bond-breaking agents are available, but the most widely used varieties are substituted mercaptans, 2% to 10% thioglycolates, mixed with 2% to 6% of either NaOH or CaOH. The sulfide depilatories agents are faster acting but are more irritating. They produce an undesirable odor of hydrogen sulfide gas and are poisonous if ingested. Sodium hydroxide, also known as lye, is the best bond breaking agent but is extremely damaging to the skin. Depilatories are available in pastes, creams, and lotions. Formulations are specially adapted for the use on legs, bikini area, and face. A depilatory labeled for use on the legs should never be applied on the face; it is likely to be harsh. Unfortunately, skin irritation may develop after use because hair and skin are similar in the composition and any compound that has a destructive effect on hair will also affect the skin to some extent. The product should not be used in patients with any kind of dermatological problems. Both irritant and allergic contact dermatitis can occur with the use of chemical depilatories. The latter is rare and may be related to fragrances, lanolin derivatives, or the thioglycolate. Darkly pigmented hair seems somewhat more resistant to removal than lighter hair, and coarse hair is more resistant than fine hair.

Pumice stones are among the oldest devices employed for temporary hair removal, but also the least popular. This stone is a gray, solid, soft, and porous material of volcanic origin. The other alternative is a depilatory glove. It is made of fine sandpaper shaped into the form of a mitten. Rubbing it over the skin in circular movements, after cutting the hair as short as possible, at the site of hair growth produces mechanical friction, which wears off the hairs at the surface of the skin. This can remove hair doser to the surface of the skin than shaving does. In spite of that, it is a temporary method since it does not affect the hair roots. They are cheap, and easy to use. Disadvantages of using abrasives are the slow and tedious application as well as its impracticability for large areas. The potential for irritation is alway present, especially if the abrasive is rubbed too vigorously, the skin is almost always irritated and with time lichenification of the area may occur. After the use of abrasives, a mild emollient cream should be applied to the area to relieve any kind of skin irritation.

Electrolysis involves inserting a fine needle into the hair follicle and applying an electrical current to the follicle root. This procedure actually burns the hair root theoretically preventing it from producing more hair. The current induces heating of water by molecular vibration, which subsequently destroys the follicular germinative cells. If the tissues have been adequately damaged to prevent hair regrowth, the hair shaft can be easily plucked with tweezers. Each hair follicle must be treated individually and may take several treatments to destroy the follicle. Electrolysis is a permanent form of hair removal but it has several drawbacks. First, there are no standardized licensing guidelines for electrolysis so finding an experienced, effective technician is difficult. Second, this method requires repeated treatments for up to 12 to 18 months. Hair follicles that are in the resting phase are more difficult to destroy than hair follicles in the anagen or growth phase. Shaving approximately three days before an electrolysis treatment ensures that the hairs that are visible are in the anagen phase. Finally, side effects can include pain, infection, and possibly keloid formation.

Laser treatment of various skin conditions has blossomed, as laser technology has become more understood. Hair removal is a common application of laser technology, but not all laser treatments are permanent and not for everyone. Lasers work by emitting light at various wavelengths, energy output, and pulse widths. The wavelength used determines the skin structure it will affect such as veins, melanin, or water. Most lasers used for hair removal target melanin and are therefore designed to burn structures that contain melanin. The more melanin, the more damage. It makes sense that laser hair removal works best for light-skinned people with dark hair. As with electrolysis, hair follicles in the anagen phase are more easily destroyed than those in the telogen phase. Therefore, laser treatments for hair removal must be repeated. At this time it appears that laser treatment, while not causing permanent destruction of all hair follicles, does retard the regrowth of new hair. Lasers respond to melanin from any source, so must be very carefully controlled or burning can result, especially on tanned or darker skin.

One successful method for treating persons suffering from hair removal skin irritation side effects involves the use of a frozen compress impregnated with milk. Milk removes acid from the skin by neutralizing skin pH thereby enabling the skin to heal sooner and be less uncomfortable. The cold decreases the swelling and also gives immediate relief as a result of the numbing effect of the cold application. The problem however with this treatment has been that most persons find this too messy and inconvenient because it can only be used at home and they would like something to use “on the go” without the mess and preparation. If the technique were able to be used intermittently all day long the response which is relief from pain and irritation would be more rapid and effective.

We have discovered a single type of treatment for skin irritation which is caused by hair removal by any of the aforesaid methods and which works for all parts of the body. This treatment works no matter what the technique used is for the hair removal.

### DISCLOSURE OF THE INVENTION

We have designed various pads that can hold a source of cold, which pads are coated with a stable milk product The pads can be used in the different parts of the body where hair has been removed by various techniques. These portable cold pads are to be used immediately after the hair removal treatments and their use can be continued until relief has occurred. Immediate use of the pads will help prevent the inflammation and discomfort following hair removal. If the pads are used both immediately and on an ongoing basis until the irritation subsides, they will offer many advantages such as enabling patients to wear their normal clothing and not be restricted to loose fitting items during recovery, and will let them carry on normal activities while the healing process occurs. They will also allow women who have had hair removal in the pubic area to return to normal sexual relations sooner and will allow the users to return to physical exercise sooner in that perspiration can be irritating to these post procedure areas and often needs to be avoided. They will also allow women to use facial makeup sooner in that the areas are quite sensitive post procedure.

Men will find healing of both backs and chest will be much more rapid therefore allowing them to get back to normal activities sooner.

Other advantages of the method and devices of this disclosure is that they will promote more rapid healing, will also decrease opportunities for infections to occur, and will be much more likely to be used consistently.

The following are body areas which experience irritation from various hair removal procedures, and some proposed products which could be used for treating such irritation and pain resulting from hair removal.

1. Pubic area: A triangular shaped pad that is coated with milk. This pad will be constructed such that it will have a pouch to contain a reusable and freezable gel pack. Another useful product would be a design that will contain a cold pack using an endothermic system (instant) that can be activated to be used on the go. On one side it could also have an adhesive strip to stick to the underwear as do mini pads

2. Pubic/vulva: A T-shaped or triangular pad in both reusable and instant versions.

3. Legs: Pads which can be wrapped around the legs with VELCRO, or other fastening devices, in either single use or reusable multi-use versions.

4. Back/chest/buttocks: Pads that are made in squares in both versions. In that rarely is an entire back or chest done at the same sitting, the “sectional” pads will adjust to whatever area is needed. They will have a belt or strap to hold them in place so as not to further irritate the already irritated skin.

5. Mustache/eyebrows/chin: smaller rectangular (similar in shape to a Band-Aid), or a face mask which could be used if mustache, chin and cheeks are done simultaneously.

6. Ears/Nose: Q-tip type product with gel packs at ends coated with a milk-coated fabric.

The facial products could also be used for irritation around the nose from upper respiratory infections or colds or around the lips from cold sores.

The vulva products would also be very effective for post operative treatment after the removal of vaginal warts by hyfrecation or laser treatment. All of the above products utilize a combination of dry milk and a source of cold.

The aforesaid products will incorporate milk ingredient that, when applied to the irritated issue will lower the acidity of the issue and, because of other materials such as proteins in the milk, soothe the inflamed area. They will typically take the form of pads which can contain a refrigerant that can be made cold by crushing, or they can contain a gel that can be cooled by being placed in a refrigerator or freezer. The pads have the specific configurations noted above. The milk component can be incorporated into the pads in a number of different ways. For example it has been found that slurries of nonfat dry milk in water can be formed, sprayed onto the pads, dried and then are suitable for use, alternatively, slurries of nonfat dry milk and a meltable anhydrous water-soluble carrier such as polyethylene glycol or a non-meltable water-soluble carrier, such as glycerin, can be formed. Alternatively, a slurry of nonfat dry milk and a non-water-soluble carrier such as mineral oil and wax can be formed. A slurry of nonfat dry milk and an aqueous solution of thickeners and polymers can be formed. A preferred embodiment of the solution is a slurry of non fat dry milk in glycerin, either anhydrous or glycerin containing up to about 15% water, not followed by drying. The reason for this is that drying is a separate step and not all converters have the capability of drying. Additionally, it is less expensive to do one pass, which consists of doping the non-woven material, cutting it, and sealing the resultant pads in packaging.

The aforesaid dry milk can be substituted with non-dry milk, and whole milk, both liquid and dry can be used in place of the nonfat milk component. Nonfat dry milk is preferred. When a liquid milk product, either whole or nonfat, is used, the milk product would be dried after being applied to the pads. Any combination of the dry or liquid milk components, either nonfat, or whole milk, can be used in producing the pads. The milk coating can be applied to the pad cover by a doctor blade, by rollers, or by spraying. When an aqueous solution of the milk component is used, the coated pad or pad cover must be dried before the pad is ready for use. Drying can be accomplished either by forced air drying or by direct application of heat through forced hot air, heated rollers, bars or plates. The pad assembly can include the absorbent filler pad, polymers, such as high molecular weight acrylics, commonly referred to as “super slurpers”, to hold moisture and may also include a pouch containing a freezable liquid which can be frozen and slipped into the pad.

The finished pad assemblies are used in the following manner. When the milk is applied to an outer non-woven pad cover, the dried milk constituent will be in intimate contact with the subject's skin. It should be realized that when the milk slurry is applied to or dried on the pad's outer surface, the concentration of milk on the pads will be more highly concentrated than if moist milk were to be used without either super concentrating in soluble carrier or drying the milk coating. Thus, the natural moisture of the skin will dissolve or release the fat-free or whole milk to the skin. This result can be accelerated by having a semi-permeable sheet material between the outer non-woven cover and the interior of the pads. The cover serves to prevent transepidermal moisture from bypassing the dried milk, and ensures that the transepidermal moisture will solubilize the dried milk solids into a milk solution or mixture. The use of such a sheet material will increase the concentration of transepidermal water vapor such that the water vapor will enhance release of milk to the irritated areas.

In the case of application of the milk constituent to the assembled pads, the concentration of milk on the exterior and interior of the pads will depend on how it is applied, i.e., by cylinder or by spray. Use of a cylinder which directly contacts the nonwoven sheet material to coat the sheet material will result in a greater concentration of milk on the sheet material due to the direct contact that occurs between the cylinder and the sheet material. The concentration of milk in the pads will be able to be thus controlled along with the formulation type, i.e., either hydrophobic or hydrophilic, to affect both instant and/or sustained release of milk from the pads to the irritated area. A hydrophilic formulation will more readily result in release of the milk for the pad assemblies because of the transepidermal moisture. If the formulation is hydrophilic, milk release can be slowed by increasing the molecular weight of the incorporated material. Polyethylene glycol and glycerin are examples of such materials. If the material is hydrophobic, materials such as silicone can be used to retard the release of the milk constituent. Thus, the pad assemblies can be customized to a certain degree regarding the rate of release of the milk constituent.

A preferred embodiment of a pad assembly could employ an accelerated release of the milk constituent initially, followed by a slower, more sustained release of the milk constituent. The use of a polyethylene glycol matrix which includes different molecular weight fractions or the use of hydrophilic and hydrophobic carriers would achieve this desired result.

The pad assemblies can be impregnated with the milk component in a number of other ways which do not involve the formation of a milk slurry per se. One way would be to spray or coat the pad material with water, or an aqueous slurry of a sticky substance, and then sprinkle dry milk on the wet pad material. The sprinkled pad material would then be dried. One could also spray or coat the pad material with a concentrated solution or slurry or a non-concentrated solution of wet milk and then dry the milk-coated material. Another way to produce the milky material would be to add milk to a solid polymer which is to be used to form the non-woven component of the pad assembly after the polymer is melted by before it is converted into a fiber form, and then run the polymer-milk mixture through a spinneret so that the milk is incorporated into the polymer fiber.

With all embodiments of the pad assemblies, when the assemblies are worm, the milk impregnated component will be disposed against the irritated tissue. This allows transepidermal moisture to penetrate the milk impregnated component so as to moisten the dried milk in the component. It will be readily appreciated that the pad assemblies described above provide a simple, reliable and convenient treatment whereby hair removal skin irritation can be relieved. Some of the pad assemblies can be worn during most common daily activities and are not restricted to in house usage. The pad assemblies are not messy to use, and extended use of them will not result in any adverse effects to the user.

Since many changes and variations of the disclosed embodiments of the invention may be made without departing from the inventive concept, it is not intended to limit the invention otherwise than as required by the appended claims.

What is claimed is:

1. A method for reducing skin irritation resulting from hair removal, said method comprising:
a) the step of providing a pad assembly for covering the irritated skin, which pad assembly can be worn during everyday activity, said pad assembly comprising:
i) an absorbent pad component;
ii) a cooling component; and
iii) a milk-containing component; and
b) the step of positioning said pad assembly on a user's skin in a manner that will bring said milk-containing component into direct contact with irritated skin tissue of the user so as to facilitate release of said milk component onto the irritated tissues as a result of said milk component being moistened by secretions from the irritated skin.

2. The method of claim 1 wherein said pad assembly further comprises a cover component in which said absorbent pad component is disposed.

3. The method of claim 1 wherein said cooling component is a freezable substance.

4. The method of claim 1 wherein said cooling component is a gel.

5. The method of claim 1 wherein said cooling component is an endothermic substance.

6. The method of claim 1 wherein said milk component is nonfat milk.

7. The method of claim 1 wherein said milk component is formed from a slurry of dry milk and a meltable anhydrous water-soluble carrier.

8. The method of claim 7 wherein said meltable anhydrous water-soluble carrier is polyethylene glycol.

9. The method of claim 1 wherein said milk component is formed from a slurry of dry milk and a non-water soluble carrier.

10. The method of claim 1 wherein said non-water soluble carrier is mineral oil and wax.

11. The method of claim 1 wherein said absorbent pad component is disposed in a cover component, said cover being formed from a non-woven fibrous material, and said milk component being in the form of a dried coating on said cover.

12. The method of claim 11 wherein the cover component is formed from a solid polymer into which the milk component is incorporated prior to conversion of the polymer to a fibrous form.

13. The method of claim 1 wherein said milk component is a solution or slurry formed from dry milk and water.

14. The method of claim 1 wherein said milk component is formed from a slurry of dry milk and glycerin.

15. The method of claim 14 wherein said glycerin is anhydrous.

16. The method of claim 14 wherein said glycerin contains up to about fifteen percent water.
